(19) United States
(12) Patent Application Publication
(10) Pub. No.: US 2014/0235492 A1
(43) Pub. Date: Aug. 21, 2014

(54) METHODS FOR PREPARING SINGLE DOMAIN ANTIBODY MICROARRAYS

(71) Applicants: Daniel Baty, Marseille (FR); Patrick Chames, Marseille (FR); Klervi Even-Desrumeaux, Marseille (FR)

(72) Inventors: Daniel Baty, Marseille (FR); Patrick Chames, Marseille (FR); Klervi Even-Desrumeaux, Marseille (FR)

(73) Assignees: Institut National de la Sante et de la Recherche Medicate (INSERM), Paris (FR); UNIVERSITE D'AIX MARSEILLE, Marseille (FR)

(21) Appl. No.: 14/345,660

(22) PCT Filed: Sep. 20, 2011
(86) PCT No.: PCT/IB2011/002583

§ 371 Date: Mar. 19, 2014

(51) Int. Cl. G01N 33/531; G01N 33/543

(52) U.S. Cl. CPC G01N 33/531; G01N 33/54353
USPC 506/9; 506/26; 506/18

(57) ABSTRACT

The present invention relates to a method for preparing sd Ab microarray comprising the step consisting of: —i) providing a host cell capable of expressing a biotinylation enzyme —ii) transforming said host cell with a nucleic acid encoding for a fusion protein wherein a single domain antibody is fused at its carboxy terminal end to a biotinylation peptide —iii) culturing said host cell in presence of biotin in such a way that said fusion protein and biotinylation enzyme are expressed, resulting in biotinylation of said fusion protein —iv) lysing said host cell as cultured at step iii) —v) spotting the lysate obtained at step iv) on a solid sup port coated with an agent selected from the group consisting of avidin, streptavidin and/or any art known derivative of these agents A further object of the invention relates to a sd Ab microarray obtainable by the method of the invention.

### FIELD OF THE INVENTION

The present invention relates to methods for preparing single domain antibody (sdAb) microarrays and uses thereof.

### BACKGROUND OF THE INVENTION

Analytical microarrays are typically used to profile a complex mixture of proteins in order to measure binding affinities, specificities and protein expression levels. In this technique, monoclonal antibodies or derived formats such as Fab (Fragment Antigen Binding), scFv (single chain variable Fragment) but also aptamers and affibodies (Renberg et al, 2007) are arrayed on a support and the array is probed with a protein solution. Antibody microarrays, pioneered by MacBeath and Schreiber (MacBeath and Schreiber, 2000) and Haab et al (Haab et al, 2001), are the most common analytical microarray. This type of microarray will provide new means to perform differential protein expression profiling of healthy vs. diseased samples that will play a key role within disease diagnostics, biomarkers discovery and drug target identification. The ability to monitor multiple protein interactions in parallel has many advantages such as saving of time, cost, sample consumption, especially if assays are miniaturized. Most array-based strategies use sandwich assays that can be highly sensitive and specific, but this design is not compatible with high-density array. A complementary technology is label-based detection, affording high level of multiplexing and high density, despite at the expense of a lower specificity and sensitivity.

To perform global proteome analysis, high demands will be placed upon the choice of catcher proteins. The specificity of the probes is also a critical feature since analytes must be specifically detected in heterogeneous mixtures containing more than 10 000 different irrelevant proteins. Actually, only low-density antibody microarrays (on planar substrate or on bead) have successfully been designed and developed (Miller et al, 2003; Carlsson et al 2008; Ingvarsson et al, 2008; Sauer et al, 2008; Lyon et al, 2008). In contrast to nucleic acids, antibodies and proteins in general are chemically and structurally much more complex, heterogeneous, and often unpredictable regarding their interaction profiles. Therefore, it is difficult to define general protein detection and immobilization strategies that do not discriminate between proteins.

Recombinant antibody libraries such as scFv or Fab, providing numerous probes based on a single scaffold with similar biological properties, will display significant advantages. But, recombinant antibody formats such as scFv are often unstable (Honegger, 2008) and produced with a poor yield.

In 1993, Hamers-Casterman et al discovered that serum of camels, dromedaries and llamas contain a unique type of antibodies devoid of light chains. Camelids produce functional antibodies devoid of light chains (HCAbs) and CH1 domain, of which the single Nterminal domain is fully capable of antigen binding. When they are recombinantly produced, these single domain antibody fragments (sdAbs) have several advantages for biotechnological applications thanks to their unique properties of size (15 kDa), stability even without disulfide bond formation, (Gueorguieva et al, 2006), solubility, and expression yield (Muyldermans, 2001). However; use of sdAbs has not yet been investigated for preparing DNA microarray.

### SUMMARY OF THE INVENTION

The present invention relates to a method for preparing sdAb microarray comprising the step consisting of:
i) providing a host cell capable of expressing a biotinylation enzyme
ii) transforming said host cell with a nucleic acid encoding for a fusion protein wherein a single domain antibody is fused at its carboxy terminal end to a biotinylation peptide
iii) culturing said host cell in presence of biotin in such a way that said fusion protein and biotinylation enzyme are expressed, resulting in biotinylation of said fusion protein
iv) lysing said host cell as cultured at step iii)
v) spotting the lysate obtained at step iv) on a solid support coated with an agent selected from the group consisting of avidin, streptavidin and/or any art known derivative of these agents

A further object of the invention relates to a sdAb microarray obtainable by the method of the invention.

### DETAILED DESCRIPTION OF THE INVENTION

The inventors have generated proof-of-principle for several immobilization strategies of sdAbs contained in crude bacterial lysate, namely immobilization of in vivo biotinylated sdAb by direct spotting of bacterial lysate on streptavidin. By use of these immobilization strategies, the inventors compared different detection methods, either by sandwich or label-based detection. These methods allow the specific and sensitive detection of subnanomolar antigen concentration without using signal amplification in model systems with pure antigen as well as crude patient sera. They demonstrated that said methods allow a stong and oriented immobilisation of the sdAbs on the microarray. Finally, some of these sdAbs were used to elaborate a sensitive, specific, fast and efficient multiplexed assay on cytometric bead array to analyze a complex breast cancer representative sample.

The present invention relates to a single domain antibody microarray and methods for preparing thereof.

The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

As used herein, the term “single domain antibody microarray” or “sdAb microarray” encompasses a solid surface to which single domain antibodies are fixed to a solid surface. The term “single domain antibody microarray” is further meant to encompass devices that utilize immobilized single domain antibodies as capture probes.

More particularly, the present invention relates to a method for preparing sdAb microarray comprising the step consisting of
i) providing a host cell capable of expressing a biotinylation enzyme
ii) transforming said host cell with a nucleic acid encoding for a fusion protein wherein a single domain antibody is fused at its carboxy terminal end to a biotinylation peptide
iii) culturing said host cell in presence of biotin in such a way that said fusion protein and biotinylation enzyme are expressed, resulting in biotinylation of said fusion protein
iv) lysing said host cell as cultured at step iii)
v) spotting the lysate obtained at step iv) on a solid support coated with an agent selected from the group consisting of avidin, streptavidin and/or any art known derivative of these agents

The term “host cell” refers to a eukaryotic or procaryotic cell or group of cells that can be or has been transformed by a recombinant DNA vector. For purposes of the present invention, procaryotic host cells are preferred. Typically a host cell according to the invention is E. coli.

The term “biotinylation enzyme” refers to the class of enzymes known as biotin protein ligases, or enzymes which biotinylate other proteins or peptides. Biotinylation enzymes are well known in the art but typically a biotinylation enzyme according to the invention is BirA of E. Coli as described in O'callaghan CA, Byford M F, Wyer J R, Willcox B E, Jakobsen B K, McMichael A J, Bell J I. BirA enzyme: production and application in the study of membrane receptor-ligand interactions by site-specific biotinylation. Anal Biochem. 1999 Jan. 1; 266(1):9-15.

According to the invention the host cell can naturally express the biotinylation enzyme. Alternatively, the host cell may be previously transformed with a nucleic acid encoding for the biotinylation enzyme (eg. A BirA plasmid).

In a particular embodiment, the host cell is E. coli, and the biotinylation enzyme is BirA.

The term “fusion protein” generally refers to a protein which is a composite of two separate proteins which are normally not fused together as a single protein. According to the invention fusion proteins are prepared by recombinant nucleic acid methods, i.e., as a result of transcription and translation of a gene fusion comprising a segment which encodes a single domain antibody and a segment which encodes a biotinylation peptide.

The sdAb according to the invention and the biotinylation peptide may fused directly of via a spacer.

As used herein, the term “directly” means that amino acid at the C-terminal end of the sdAb is fused to the amino acid at the N-terminal end of the biotinylation peptide.

As used herein, the term “spacer” refers to a sequence of at least one amino acid that links the sdAb with the biotinylation peptide. Typically, said spacer is an amino acid sequence having less than 20 amino acids. The skilled man in the art can easily select the appropriate spacer. Typically a spacer according to the invention can be the his6-Tag as described in the EXAMPLES.

According to the invention, the sdAb may be directed against any antigen.

For example, the sdAb according to the invention may be directed against a cancer antigen. Known cancer antigens include, without limitation, c-erbB-2 (erbB-2 is also known as c-neu or HER-2), which is particularly associated with breast, ovarian, and colon tumor cells, as well as neuroblastoma, lung cancer, thyroid cancer, pancreatic cancer, prostate cancer, renal cancer and cancers of the digestive tract. Another class of cancer antigens is oncofetal proteins of nonenzymatic function. These antigens are found in a variety of neoplasms, and are often referred to as “tumor-associated antigens.” Carcinoembryonic antigen (CEA), and α-fetoprotein (AFP) are two examples of such cancer antigens. AFP levels rise in patients with hepatocellular carcinoma: 69% of patients with liver cancer express high levels of AFP in their serum. CEA is a serum glycoprotein of 200 kDa found in adenocarcinoma of colon, as well as cancers of the lung and genitourinary tract. Yet another class of cancer antigens is those antigens unique to a particular tumor, referred to sometimes as “tumor specific antigens,” such as heat shock proteins (e.g., hsp70 or hsp90 proteins) from a particular type of tumor. Other targets include the MICA/B ligands of NKG2D. These molecules are expressed on many types of tumors, but not normally on healthy cells.

Additional specific examples of cancer antigens include epithelial cell adhesion molecule (Ep-CAM/TACSTD1), mesothelin, tumor-associated glycoprotein 72 (TAG-72), gp100, Melan-A, MART-1, KDR, RCAS1, MDA7, cancer-associated viral vaccines (e.g., human papillomavirus antigens), prostate specific antigen (PSA, PSMA), RAGE (renal antigen), CAMEL (CTL-recognized antigen on melanoma), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), cancer-associated ganglioside antigens, tyrosinase, gp75, C-myc, Mart1, MelanA, MUM-1, MUM-2, MUM-3, HLA-B7, Ep-CAM, tumor-derived heat shock proteins, and the like (see also, e.g., Acres et al., Curr Opin Mol Ther 2004 February, 6:40-7; Taylor-Papadimitriou et al., Biochim Biophys Acta. 1999 Oct. 8; 1455(2-3):301-13; Emens et al., Cancer Biol Ther. 2003 July-August; 2(4 Suppl 1):S161-8; and Ohshima et al., Int J Cancer. 2001 Jul. 1; 93(1):91-6). Other exemplary cancer antigen targets include CA 195 tumor-associated antigen-like antigen (see, e.g., U.S. Pat. No. 5,324,822) and female urine squamous cell carcinoma-like antigens (see, e.g., U.S. Pat. No. 5,306,811), and the breast cell cancer antigens described in U.S. Pat. No. 4,960,716.

The sdAb according to the invention may target protein antigens, carbohydrate antigens, or glycosylated proteins. For example, the sdAb can target glycosylation groups of antigens that are preferentially produced by transformed (neoplastic or cancerous) cells, infected cells, and the like (cells associated with other immune system-related disorders). In one aspect, the antigen is a tumor-associated antigen. In an exemplary aspect, the antigen is O-acetylated-GD2 or glypican-3. In another particular aspect, the antigen is one of the Thomsen-Friedenreich (TF) antigens (TFAs).

The sdAb according to the invention can also exhibit specificity for a cancer-associated protein. Such proteins can include any protein associated with cancer progression. Examples of such proteins include angiogenesis factors associated with tumor growth, such as vascular endothelial growth factors (VEGFs), fibroblast growth factors (FGFs), tissue factor (TF), epidermal growth factors (EGFs), and receptors thereof; factors associated with tumor invasiveness; and other receptors associated with cancer progression (e.g., one of the HER1-HER4 receptors).

Alternatively the sdAb according to the invention can be specific for a virus, a bacteria or parasite associated target. For example, the sdAb may be specific for a virus-associated target such as an HIV protein (e.g., gp120 or gp41), CMV or other viruses, such as hepatitis C virus (HCV).

sdAbs are usually generated by PCR cloning of the V-domain repertoire from blood, lymph node, or spleen cDNA obtained from immunized animals into a phage display vector, such as pHEN2. Antigen-specific sdAbs are commonly selected by panning phage libraries on immobilized antigen, e.g., antigen coated onto the plastic surface of a test tube, biotinylated antigens immobilized on streptavidin beads, or membrane proteins expressed on the surface of cells. However, such sdAbs often show lower affinities for their antigen than sdAbs derived from animals that have received several immunizations. The high affinity of sdAbs from immune libraries is attributed to the natural selection of variant sdAbs during clonal expansion of B-cells in the lymphoid organs of immunized animals. The affinity of sdAbs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). sdAbs derived from camelid are readily expressed in and purified from the E. coli periplasm at much higher levels than the corresponding domains of conventional antibodies. sdAbs generally display high solubility and stability and can also be readily produced in yeast, plant, and mammalian cells. For example, the “Hamers patents” describe methods and techniques for generating VHH against any desired target (see for example U.S. Pat. No. 5,800,988; U.S. Pat. No. 5,874,541 and U.S. Pat. No. 6,015,695). The “Hamers patents” more particularly describe production of sdAbs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

The term “biotinylation peptide” refers to an amino acid sequence which provides a biotinylatable sequence motif. Thus, a biotinylation peptide is a peptide that is capable of being biotinylated.

In some embodiments, the biotinylation peptide is a BirA substrate sequence tag. A BirA substrate sequence tag according to the invention is defined herein as a peptide sequence present in a polypeptide that provides a specific site for BirA to biotinylate the peptide substrate. Many BirA substrate sequence tags are known to the art. Typically, such sequences exhibit a common structure, which preferably contains the amino acid motif AMKM (SEQ ID NO: 1) or certain variations thereof. In addition, there exist peptide sequences which do not contain this consensus sequence, but can also be biotinylated by biotin protein ligases (Schatz, P. J., Biotechnology 11 (1993) 1138-1143, incorporated by reference herein). Typically, BirA substrate sequence tags have a length of about less than 50 amino acids, and most preferably a length of about 10 to 20 amino acids. Typically, a BirA substrate sequence tag according to the invention is the 15 amino acid peptide tag AviTag™ commercially available from Avidity, Inc., Indianapolis, Ind.; the sequence of which is GLNDIFEAQKIEWHE (SEQ ID NO:2). Additional examples of polypeptide sequences which can be biotinylated enzymatically and site-specifically are also described in Cronan, J. E., Jr., et al., J. Biol. Chem. 265 (1990) 10327-10333; and Samols, D., et al., J. Biol. Chem. 263 (1988) 6461-6464, all of which are incorporated by reference herein. Further examples are shown in U.S. Pat. Nos. 5,723,584; 5,874,239; and 5,932,433, all of which are incorporated by reference herein.

The nucleic acid encoding the fusion protein of the invention can be obtained by conventional methods well known to those skilled in the art.

Typically, said nucleic acid is a DNA or RNA molecule, which may be included in a suitable vector, such as a plasmid, cosmid, episome, artificial chromosome, phage or viral vector.

So, a further object of the present invention relates to a vector and an expression cassette in which a nucleic acid molecule encoding for an antigen binding format of the invention is associated with suitable elements for controlling transcription (in particular promoter, enhancer and, optionally, terminator) and, optionally translation, and also the recombinant vectors into which a nucleic acid molecule in accordance with the invention is inserted. These recombinant vectors may, for example, be cloning vectors, or expression vectors.

As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like.

Other examples of plasmids include replicating plasmids comprising an origin of replication, or integrative plasmids, such as for instance pUC, pcDNA, pBR, and the like.

Other examples of viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

Any source of biotin may be used. Biotin can be added to the culture medium in a concentration ranging from 1 to 100 μM, and more preferably at a concentration of about 5004.

After the expression of the fusion polypeptide, biotinylation occurs under standard reaction conditions, preferably within 10 to 30 hours at 20° C. to 36° C., most preferably at about 30° C.

Time for allowing production of sdAb and biotinylation thereof is comprised between 1 h and 10 h, but 3 h may be sufficient as described in the EXAMPLE 1.

Any method well known in the art for lysing host cell may be suitable. Typically, host cells may be lysed as described in the EXAMPLE 1.

According to the invention it is not necessary to purify the host cell lysates before spotting the fusion protein on the solid support, but optionally, lysates containing biotinylated fusion proteins produced according to the present invention can be further purified by any well known method in the art.

The skilled man in the art can easily select the amount of lysate that shall be deposited in the solid support. Typically, amounts less than 100 nL are sufficient, preferably 50 nL.

The term “solid support” refers to a material having a rigid or semi-rigid surface. Such materials will preferably take the form of small beads, pellets, disks, chips, or wafers, although other forms may be used. Such surfaces include, simply by way of example, surfaces of art-known supports such as beads, plates, cuvettes, filters, titer plates, and the like, that have avidin, streptavidin and/or any art known derivative of these agents linked or coated to the surface(s) of those supports. The supports are generally made of conventional materials, e.g., plastic polymers, cellulose, glass, ceramic, stainless steel alloy, and the like.

It is also contemplated that modified forms of avidin or streptavidin are employed to bind or capture polypeptides biotinylated by the methods of the invention. A number of modified forms of avidin or streptavidin that bind biotin specifically are known. Such modified forms of avidin or streptavidin include, e.g., physically modified forms (Kohanski, R. A. and Lane, M. D. (1990) Methods Enzymol. 194-200), chemically modified forms such as nitro-derivatives (Morag, E., et al., Anal. Biochem. 243 (1996) 257-263) and genetically modified forms of avidin or streptavidin (Sano, T., and Cantor, C. R., Proc. Natl. Acad. Sci. USA 92 (1995) 3180-3184).

Methods for coating biotinylated polypeptides on a solid support coated with avidin, streptavidin and/or any art known derivative of these agents are well known in the art. Typically, said immobilization may be performed as described in the EXAMPLE.

Once immobilization is done, the solid support may be washed and optionally dried.

A further object of the invention relates to a sdAb microarray obtainable by the method of the invention.

In some embodiments, the microarray obtainable by the method of the invention has a density of at least 5 spots/cm², preferably at least 10, 20, 30, 40, 50, 60, 70, 80, 90, 100, 110, 120, 130, 140, 150, 160, 170, 180, 190, 200, 210, 220, 230, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000 or 9000 spots/cm².

In some embodiments the microarray obtainable by the method of the invention is spotted with sole type of sdAb directed against the same antigen, or with different kinds of sdAbs directed against various antigens.

In a particular embodiment, the solid support of the sdAb array of the invention is a cytometric bead for use in flow cytometry. Such beads may for example correspond to BD™ Cytometric Beads commercialized by BD Biosciences (San Jose, Calif.). Typically sdAb arrays based on cytometric bead may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or uv laser (e.g. Pacific blue, pacific orange).

In another particular embodiment, the solid support of the sdAb array of the invention is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof.

In another particular embodiment, the solid support of the sdAb array of the invention is bead that is dyed and magnetized.

The microarray obtainable by the method of the invention can be used for example as a diagnostic and as a tool for antigen profiling of a given source. The microarray obtainable by the method of the invention can also be used to find a different antibody (i.e sdAb) for an antigen which has one or more antibodies already, but for which another antibody might be desirable to identify. For example, where a given previously identified antibody will not work well as a therapeutic or diagnostic antibody, it would be desirable to find another antibody for that target antigen that could perhaps work well as a therapeutic or diagnostic antibody. The microarray obtainable by the method of the invention can also be used to compare antigen profiles from two or more comparable sources of antigen. For example, a normal tissue source can be compared to a diseased tissue source in order to identify antigen differences, or antigen profiles, or the two or more sources. The method is particularly suitable for identifying new cancer antigens.

In some embodiments, the present invention provides methods for detecting the antigens bound to the microarray obtainable by the method of the invention. Briefly, the methods comprise the steps of providing sdAb array according to the invention, contacting the array with a sample containing antigens, and detecting the bound antigens. The process can be done manually and/or automatically. The handling of arrays is well known to those skilled in the art.

As used herein, the term “sample” encompasses a variety of sample types and/or origins, such as blood and other liquid samples of biological origin (e.g. urine), solid tissue samples such as a biopsy specimen or tissue cultures or cells derived therefrom, and the progeny thereofhe term “sample” encompasses a clinical sample, and also includes cells in culture, cell supernatants, cell lysates, serum, plasma, biological fluid, and a pure or enriched bacterial or viral sample derived from any of these, for example, as when a sample is cultured in order to increase, enrich and/or substantially purify a bacterial or viral sample therefrom. A sample can be from microorganisms, e.g., bacteria, yeasts, viruses, viroids, molds, fungi, plants, animals, including mammals such as humans. A sample may comprise a single cell or more than a single cell. These samples can be prepared by methods known in the art such as lysing, fractionation, purification, including affinity purification, FACS, laser capture microdissection or iospycnic centrigugation.

When the sdAb array is contacted with a sample, the antigen complexes can be performed under a variety of conditions. Typically, the reaction solutions can contain varying degrees of salt or have varying pH values. In addition, the binding reaction can be carried out at varying temperature. In general, pH conditions will range from 2-10 (most preferably around pH7), temperatures from 4-45° C. (preferably 15-30° C.) and salt conditions from 1 μM to 5M (in the case of NaCl).

The readout of the detecting agents bound to the sdAbs in the array of the invention can take up many forms.

Typically, the antigen can be detected with a second labelled antibody to form a sandwich assay. For example the second antibody may be labelled with any detectable molecule such as fluorescent compound known in the art. For example said fluorescent compound include FITC (FL1), PE (FL2), fluorophores for use in the blue laser (e.g. PerCP, PE-Cy7, PE-Cy5, FL3 and APC or Cy5, FL4), fluorophores for use in the red, violet or uv laser (e.g. Pacific blue, pacific orange).

In another embodiment, the antigen that shall be detected is directly labelled with a detectable molecule such as above described.

Alternatively, flow cytometry may be used, especially in a multiplexed bead assay. In said embodiment at least two bead sdAb arrays are provided (i.e. a first bead sdAb array with a sdAb directed against a first antigen of interest and a second bead sdAb array with a sdAb directed against a first antigen of interest). Flow cytometers enable the characterization of particles on the basis of light scatter and particle fluorescence. In a flow cytometer, beads are individually analyzed by exposing each particle to an excitation light, typically one or more lasers, and the light scattering and fluorescence properties of the particles are measured. Flow cytometers are commercially available from, for example, BD Biosciences (San Jose, Calif.). Analysis by flow cytometry enables both detecting the presence of bead-antigen complexes and simultaneously measuring the amount of reporter fluorescence associated with the complex as a quantitative measure of the antigen present in the sample. The simultaneous analysis of multiple antigens in a sample could be carried out using a set of distinguishable beads, each type of bead coated with a unique sdAb. The bead set and fluorescently labelled reporter reagents, one for each species of antigens to be detected, are incubated with a sample containing the antigens of interest to allow for the formation of bead-antigen complexes for each antigen present, and the resulting complexes are analyzed by flow cytometry to identify and, optionally, quantify the antigens present in the sample. Because the identity of the antigen bound to the complex is indicated by the identity of the bead, multiple antigens can be simultaneously detected using the same fluorophore for all reporter reagents.

The invention will be further illustrated by the following figures and examples. However, these examples and figures should not be interpreted in any way as limiting the scope of the present invention.

### FIGURES

FIG. 1: In Vivo Biotinylation and Multi Tags Strongly Improve Immobilization of sdAbs.

A) Serial dilutions of pure anti-Nef sdAb biotinylated in vivo (•), in vitro (▪) or unbiotinylated (▴) were coated on streptavidin plate and incubated with Nef at 5 nM. The captured antigen was detected with a mouse anti-Nef antibody followed by a goat anti-mouse HRP-conjugated mAb.

FIG. 2: Bacterial Lysates are a Good Source of Capture Antibody.

Streptavidin beads were coated with sdAb against Nef biotinylated in vivo pure (•) (1 μg/ml) or in bacterial lysate (▴) (50 nl/wells) or sdAb against Nef unbiotinylated in bacterial lysate (▾) and incubated with serial dilution of Nef. The captured antigen was detected with a mouse anti-Nef antibody followed by a goat anti-mouse HRP-conjugated mAb. Standard deviation represents two experiments performed in triplicates.

FIG. 3: In Vivo Biotinylated sdAbs in Bacterial Lysate Allow a Sensitive Antigenic Detection on Slide or Beads.

C): Streptavidin beads were coated with bacterial lysate (50 nl/wells) containing anti-Nef sdAbs biotinylated in vivo (•, ▴, ♦) or not (▪, ▾, ∘) and incubated with serial dilutions of Nef or Alexa488-conjugated Nef (♦, ∘). The captured antigen was detected with a mouse anti-Nef antibody followed by a goat anti-mouse HRP (•, ▪) or Alexa488-conjugated mAb (▴, ▾). D) Nitrocellulose slides were incubated with streptavidin (S) or PBS(Ø). Bacterial lysates containing sdAbs biotinylated in vivo or unbiotinylated were spotted. Serial dilutions of Nef were incubated and the captured antigen was detected with a mouse anti-Nef antibody followed by a goat anti-mouse Alexa705-conjugated mAb. Standard deviation represents two experiments performed in triplicates.

FIG. 4: Immobilization of In Vivo Biotinylated sdAbs Allows a Sensitive Detection of CEA in Patient Sera Using a Bead Assay.

Streptavidin beads were coated with bacterial lysates (0.5 μl/wells) containing in vivo biotinylated (•, ▪, ▾, ▴) or unbiotinylated (♦) anti-CEA sdAbs and incubated with serial dilutions of patient sera (•: 51 CEA 276 ng/ml, ▪: S2 CEA 769 ng/ml, ▴: S3 CEA 178 ng/ml, ▾: S4 CEA<5 ng/ml). The captured antigen was detected with a mouse anti-CEA antibody (35A7) followed by a goat anti-mouse HRP-conjugated mAb. Standard deviation represents two experiments performed in triplicates.

FIG. 5: Characterization of Anti Cytokeratin 19 sdAb.

A) Lysate of breast cancer cell lines (BT474, SK-BR-3, HCC1954, MCF7, MDA-MB-231, T47D, HCC1806, BRCA-Mz-01, HCC1937), healthy breast cell line (HME1), mouse cell line (MC38) patient PBMC or 12 breast cancer biopsies (5734, 5772ext, 5712, 5586, 5766, 5801, 5572int, 5592, 5627, 5011, 5033, 5713) were coated on maxisorp plates. KE9 phage-sdAb was added and bound phages were detected by HRP-conjugated aM13 mAb. B) HCC1954 cells were incubated with serial dilutions of in vitro biotinylated KE9 sdAb (•) and captured antibodies were detected by PE-conjugated streptavidin. Cells were analyzed by flow cytometry assay on MACSQuant. Error bars represent the standard deviation of experiments performed in triplicates. C) In vivo biotinylated KE9 sdAb or control sdAb were immobilized on streptavidin beads and used to perform immunoprecipitation using T47D, SKBR3 and MC38 cell line lysates. Immunoprecipitated proteins were analyzed under non-reducing conditions by SDS-PAGE.

FIG. 6: Profiling of Breast Cancer Cell Line and Biopsy Lysates by Sandwich sdAb ELISA.

In vivo biotinylated sdAb KE23 was immobilized on streptavidin plates at 10 μg/ml. Lysates of breast cancer cell line (BT474, SK-BR-3, HCC1954, MCF7, MDA-MB-231, T47D, HCC1806, BRCA-Mz-01, HCC1937), healthy breast cell line (HME1), mouse cell line (MC38) patient PBMC or 12 breast cancer biopsies (5734, 5772ext, 5712, 5586, 5766, 5801, 5572int, 5592, 5627, 5011, 5033, 5713) were added. After washing, KE32 phage-sdAb was added and bound phage were detected by HRP-conjugated aM13 mAb.

FIG. 7: Multiplexed Analysis of Four Breast Cancer Specific Targets in a Complex Sample Using a CBA Assay.

CBA beads were coated with streptavidin and incubated with in vivo biotinylated sdAb anti-HER2, anti-CEA, anti-KRT19 or sdAb KE23. All beads were mixed and incubated with serial dilution of serum containing CEA (starting dilution: 500 ng/ml), HER2-Fc antigen (starting dilution: 10 μg/ml), and biopsy 5712 lysate (starting dilution: 100 μg/ml of total protein and 7 μg/ml of KRT19). Beads were incubated with KE32 phage-sdAb, aCEA, aHER2 and aKRT19 mouse antibody. Anti M13 mAb was added, followed by PE conjugated goat-anti-mouse mAb. Beads were analyzed by flow cytometry assay on MACSQuant. Error bars represent the standard deviation of experiments performed in triplicates.

### EXAMPLE 1

### Strong and Oriented Immobilization of Single Domain Antibodies from Crude Bacterial Lysates for High-Throughput Compatible Cost-Effective Antibody Array Generation

The results reported in EXAMPLE 1 were presented in a scientific article (Even-Desrumeaux K, Baty D, Chames P. Strong and oriented immobilization of single domain antibodies from crude bacterial lysates for high-throughput compatible cost-effective antibody array generation. Mol Biosyst. 2010 November; 6(11):2241-8. Epub 2010 Sep. 21.), which is incorporated herein by reference in its entirety.

Material & Methods

Proteins and Serum Sample

Anti-HIV-1 Nef sdAb (Bouchet J, Basmaciogullari S E, Chrobak P, Stolp B, Bouchard N, Fackler 0, Chames P, Jilicoeur P, Benichou S, Baty D (2011) Inhibition of the Nef regulatory protein of HIV-1 by a single-domain antibody. Blood, 117, 3559-68 and [21]) and anti-CEA sdAb [22] were selected from immunized sdAb libraries. pET vector were used to produce in vivo biotinylated sdAbs. All sdAbs produced in this vector carry a C-terminal his₆-tag, with or without Avitag™ (GLNDIFEAQKIEWHE) (SEQ ID NO:2) upstream. To generate plasmids coding for sdAb-Avitag™-his₆, sdAb-tags was first amplified from pET-sdAbaNef-his₆ using primers birA 6 hrev (TCAGCAAGCTTAGGATCCGTGATGATGATGGTGGTGTTCGTGCCATTCGATTTTC TGAGCCTCGAAGATGTCGTTCAGACCTGCGGCCGCTGAGGAGACAG) (SEQ ID NO:3) and seqT7 (TAATACGACTCACTATAGGG) (SEQ ID NO:4). Purified PCR product were digested with NcoI and BamHI and followed by gel purification and ligation into vector pET-sdAbaNef-his₆ which had been previously digested with the same restriction enzymes.

pJF55 vector was used for the production of sdAbs fused to c-myc tag. All sdAbs produced in this vector contain one, three, or no C-terminal myc-tag (EQKLISEEDL) (SEQ ID NO:5) followed by a his₆-tag. Vector pJF55-trimyc-his₆ was generated by overlapping PCR using primers trimy c f or (ACCGTCTCCTCAGCGGCCGCAGAACAGAAACTGATCTCTGAAGAGGACCTGAAC GGTGAGCAGAAGCTCATTTCCGAGG) (SEQ ID NO:6) and trimycrev (CGCCAAAACAGAAGCTTTTAGTTGAGGTCCTCTTCGCTGATCAATTTTTGTTCGC CATTCAAATCTTCCTCGGAAATGAGCTTCTGC) (SEQ ID NO:7). Then, the purified PCR product was digested with NotI and HindIII, gel-purified and cloned into vector pJFsdAb-cmyc-his₆ that had been digested with the corresponding restriction enzymes. All constructs were verified by nucleotide sequencing.

Patient sera were kindly provided by Pr. J. H. Cohen, (Université de Reims Champagne-Ardenne, Reims). Concentration of soluble CEA in patient sera varied between 150 and 750 ng/ml while CEA negative sera have a concentration of CEA lower than 5 ng/ml.

In Vitro Biotinylation

The in vitro biotinylation of protein was performed using Ez-link micro NMHS-PEO4-biotinylation kit (Perbio science) following the recommendation of the manufacturer.

Labeling with Alexa488

The labeling of Nef with Alexa488 was performed using Alexa Fluor 488 Microscale Protein Labeling kit (Invitrogen) following the recommendation of the manufacturer to obtain a degree of labeling (DOL) of Nef of around 3 Alexa per molecule.

Production and Purification of sdAbs

Vectors pET and pJF containing different sdAbs were transformed in B121DE3 and DH5α strain respectively. Cells containing the plasmid were inoculated in 10 ml of 2YT medium (bactotryptone 16 g/l, yeast extract 10 g/l, NaCl 85 mM) supplemented with ampiciline (100 μg/ml) and glucose (2%). Cells were grown over night at 37° C. (250 rpm). Then cells were diluted to obtain an OD₆₀₀ of 0.1 in 400 ml of 2YT medium supplemented with ampicillin (100 μg/ml) and cultures were grown until the OD₆₀₀ reached 0.5, when sdAb expression was induced by the addition of 0.1 mM IPTG (isopropyl-h-D-thiogalactopyranoside) at 30° C. (250 rpm) for 20 h. For in vivo biotinylated sdAbs, bacteria were co-transformed with pBir vector (Avidity, Colo.) and the culture medium was supplemented with chloramphenicol (50 μg/mL) during production. Fifty μM biotin was added during the induction.

Cells were harvested by centrifugation at 4000 rpm for 10 min at 4° C. For periplasmic purification, the cell pellet was suspended in 4 mL of cold TES buffer (0.2 m Tris/HCl, pH 8.0; 0.5 mM EDTA; 0.5 M sucrose), and 160 μL lysozyme (10 mg/mL) in TES buffer was added. Cells were subjected to osmotic shock by the addition of 16 mL of cold TES diluted ½ with cold H₂O. After 30 min of incubation on ice, the suspension was centrifuged at 4000 rpm for 40 min at 4° C. The supernatant was incubated with 150 μL DNaseI (10 mg/mL) and MgCl₂ (5 mM final) for 30 min at room temperature. The solution was dialyzed against 50 mM sodium acetate pH 7.0, 0.1 M NaCl, for 16 h at 4° C.

For cytoplasmic purification, cell pellet was frozen during 20 min at −80° C. and lysed by 20 ml of bugbuster (Novagen) during 20 min with low shaking.

All sdAbs were purified by affinity chromatography on Talon™ metal affinity resin (Clontech). Bound molecules were eluted with 250 mM imidazole, and proteins were concentrated in PBS by ultrafiltration with Amicon Ultra 5000 MWCO (Millipore, Billerica, Mass., USA) and stored at −20° C. Their degree of purity was evaluated by SDS-PAGE analysis and protein concentration (average of 5 mg/ml) was determined spectrophotometrically using a protein assay kit (Bio-Rad Laboratories, Hercules, Calif., USA).

Production of sdAb-Containing Crude Bacterial Lysates

Vectors pET and pJF containing different sdAbs were transformed in BL21DE3 and DH5α strain respectively. Transformed cells were inoculated in 96 well plates containing 150 μl/well of 2YT medium supplemented with ampicillin (100 μg/mL). Cells were grown until OD₆₀₀ reached 0.5 and incubated 3 hours at 37° C. after induction using 0.1 mM IPTG. For in vivo biotinylated sdAbs, bacteria were co-transformed with pBir vector and the culture medium was supplemented with chloramphenicol (50 μg/mL) during production and 50 μM of biotin was added during induction. After production, plates were centrifuged at 1700 rpm during 10 min and pellets were lysed with 30 μL of bugbuster during 20 min with low shaking Plates were stored at −20° C.

Cell Lines

MC38-CEA and MC38 [23] are a kind gift of A. Pelegrin. Cells lines were cultured in DMEM complemented with 10% (v/v) fetal calf serum at 37° C. in a humidified atmosphere and with 5% CO₂. MC38-CEA culture medium was additionally complemented with 0.5 mg/ml of geneticin.

Flow Cytometry Analysis

Experiments were performed on ice with rocking in 1% BSA PBS. Typically, 2×10⁵ cells resuspended in 50 μL were distributed in 96-well microtiter plate, and incubated for 1 h with various concentrations (500 to 0.00005 nM) of anti-CEA sdAb produced in the cytoplasm or periplasm of bacteria. After washing, binders were detected with anti-his₆ mAb (Novagen) (1:1000). Washed cells were labeled with FITC conjugated anti-mouse antibody (Jackson) (1:60). Fluorescence was measured using a FACSCalicur™ (Becton and Dickinson) and results were analysed with the Cellquest™ software. Negative (secondary antibody only) controls were carried out.

ELISA and Slide Assay

Activity of Cytoplasmic and Periplasmic sdAbs

Streptavidin plates (Thermo scientific) were blocked with 5% milk-PBS (MPBS) for two hours at RT. Fifty μl/well of biotinylated Nef at 5 nM in 2% MPBS were incubated overnight at 4° C. Wells were washed and incubated for 1 h at RT with 50 μl of 2% MPBS containing various concentrations (500 to 0.00005 nM) of anti-Nef sdAb produced in the cytoplasm or periplasm of bacteria. After three washes with PBS, plates were incubated with 9E10 mAb (against c-myc) (santa cruz biotechnology) (1 μg/ml) in 2% MPBS for one hour at RT. Following three washes with PBS, a goat anti-mouse HRP-conjugated mAb (Jackson) (0.16 μg/ml in 2% MPBS) was incubated for one hour at RT. After three washes in PBS, bound secondary antibodies were detected using ABTS. Coloration was followed at 405 nm.

Immobilization of sdAbs Biotinylated

Streptavidin plates (Thermo scientific), streptavidin beads (invitrogen), or nitrocellulose slide (Sciencetec) coated with streptavidin overnight at 4° C. (10 μg/ml) were blocked with 5% MPBS for two hours at RT. SdAbs were diluted in 50 μL of 2% MPBS and incubated overnight at 4° C. in streptavidin plate and in plate containing beads. SdAbs contain in bacterial lysate diluted ¼ in 2% MPBS were spotted and slides were dried for one hour at RT. Wells and slides were incubated with sample (Nef or serum) in 2% MPBS one hours at RT. After three washes with PBS, plates and slides were incubated with primary antibody (anti-Nef mouse mAb (kinf gift of Y. Collette, Marseille) 1:3000 or anti-CEA 35A7 antibody 2 μg/ml, (kind gift of A. Pelegrin, Montpellier) in 2% MPBS for one hour at RT. Following three washes with PBS, a goat anti-mouse HRP (Jackson) (0.16 μg/ml) or Alexa488-conjugated mAb for bead assay or Alexa680-conjugated mAb for slide assay (Invitrogen) (4 μg/ml) was incubated in 2% MPBS for one hour at RT. After three washes in PBS, plate with HRP labeled mAb was colorimetrically detected at 405 nm using ABTS substrate (Sigma), plate with Alexa labeled mAb was detected on Tristar reader (Berthold technologies) and slides with Alexa labeled mAb were read on Odyssey infrared imaging system (Licor).

Results

Domain Antibodies can be Efficiently Expressed in E. coli Cytoplasm:

Libraries of recombinant antibody fragments are a rich source of capture reagents. However, because they require disulfide bond formation, most fragments such as Fab or scFv fragments are produced in the periplasmic space of E. coli, an oxidizing environment favoring a correct folding of these fragments. In contrast, single domain antibodies are characterized by a very high solubility and stability that should allow them to fold properly in reducing environments such as the E. coli cytoplasm. To check this hypothesis, two model sdAbs (targeting Nef from HIV-1 [21] or human carcinoembryonic antigen (CEA) [22]) were produced in E. coli fused or not to a signal sequence, and purified from the periplasmic or cytoplasmic extract, respectively, and purified by metal affinity chromatography. As for most sdAbs, high production yields (10-30 mg·L⁻¹) were obtained. Gel filtration analysis showed than only monomer format was produced (data not shown). Both versions of anti-CEA sdAbs were shown to perform similarly by flow cytometry on MC38-CEA cells, a murine colon carcinoma cell line transfected with human CEA cDNA [23] and similar results were obtained with both versions of the anti-Nef sdAb by ELISA, demonstrating that sdAbs can be efficiently produced in an active form in the cytoplasm of E. coli.

Oriented sdAb Immobilization:

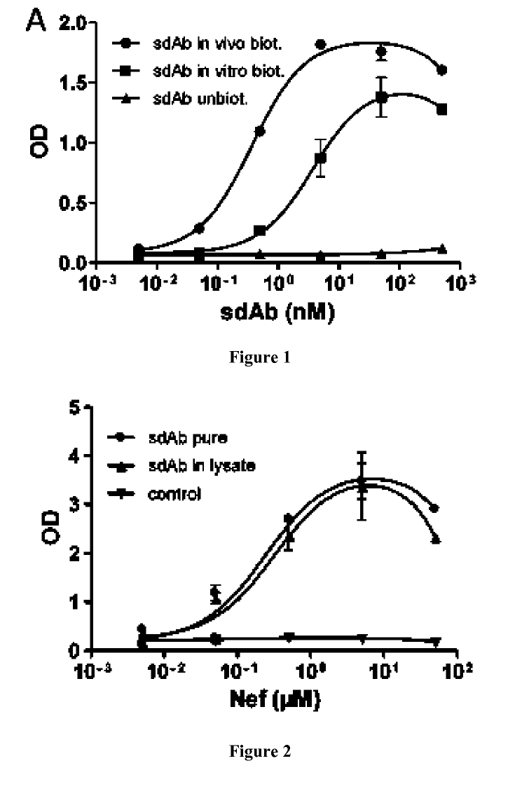

Beside its efficiency, cytoplasmic sdAb production further offers the possibility to biotinylate sdAbs in vivo using a C-terminal fusion with a 15 amino acids tag (avitag) recognized by the E. coli BirA enzyme. The resulting molecules possess a single biotin molecule coupled to a single lysine present on the avitag, which allows a near covalent and oriented immobilization through binding to streptavidin. In contrast, in vitro biotinylation can lead to inactivation of the protein and does not allow oriented immobilization. To test this hypothesis, the anti-Nef sdAb was fused to the avitag, biotinylated in vivo and purified. For comparison, the anti-Nef sdAb was purified and biotinylated in vitro using a primary amine coupling strategy. Biotinylation efficiency was checked by incubation over streptavidin beads. Up to 95% of in vivo biotinylated sdAbs and 80% of in vitro biotinylated sdAbs could be captured on beads, demonstrating an efficient biotinylation (data not shown). As shown in FIG. 1A, the in vivo biotinylated sdAb had to be diluted tenfold compared to the chemically biotinylated version to yield similar results, suggesting that in vivo biotinylation preserves the activity of the sdAb and allows an optimal orientation.

Use of Crude Lysates Containing sdAbs:

The highly efficiencies reached by these immobilization strategies allow the use of very low concentration of capture sdAbs. We reasoned that the oriented immobilization could be used as a built-in purification procedure, allowing the use of crude bacterial lysates. Indeed as demonstrated in FIG. 2, as low as 50 nL of a crude lysate containing the in vivo biotinylated anti-Nef sdAb yielded the same signal intensity as 1 μg/mL of the same purified sdAb on a bead assay, suggesting that a regular microplate sdAb production (30 μL) could be used to generate up to 600 measures.

Assay Sensitivity:

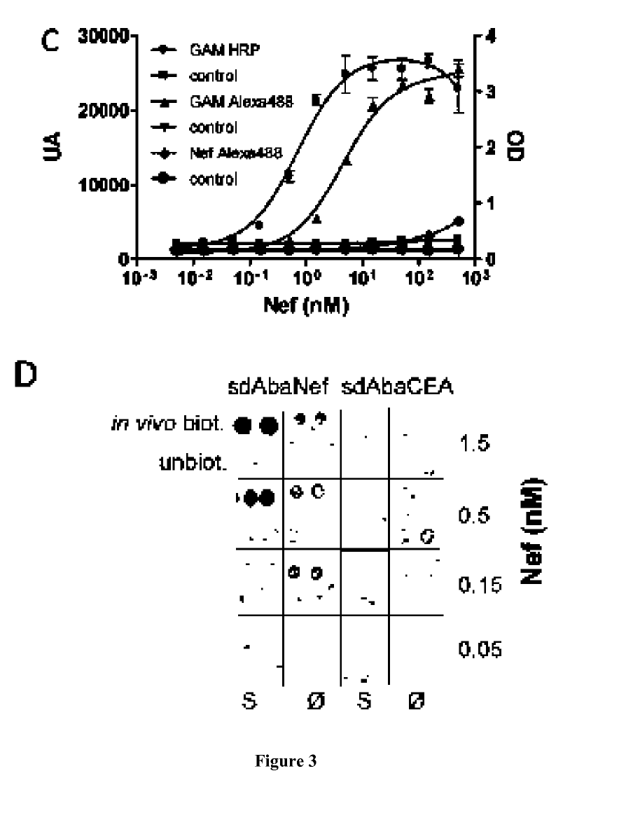

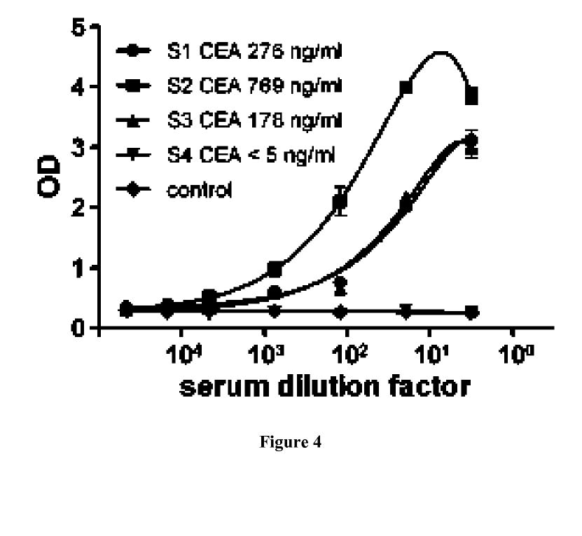

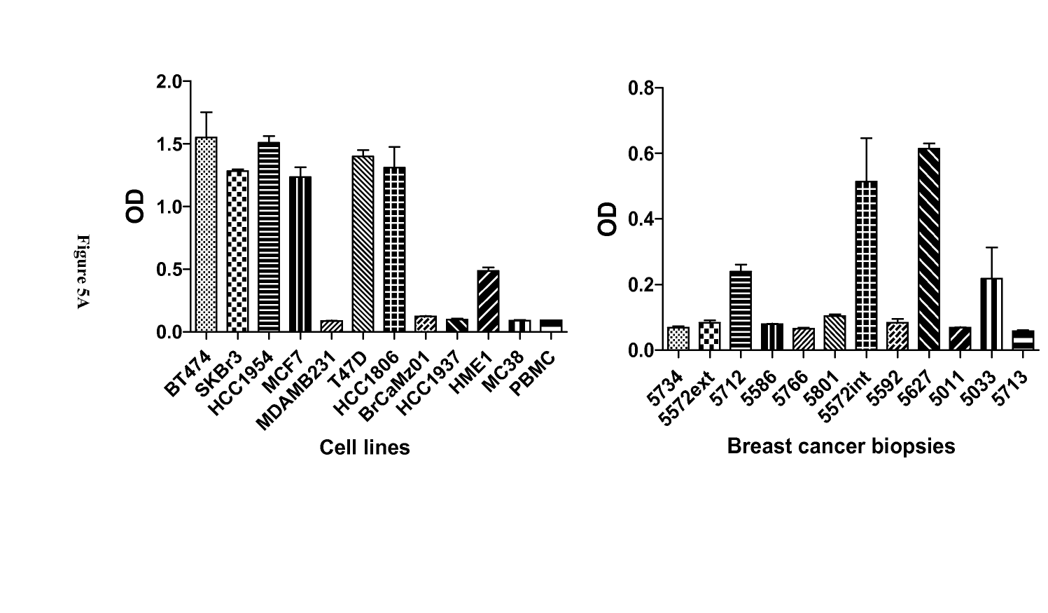

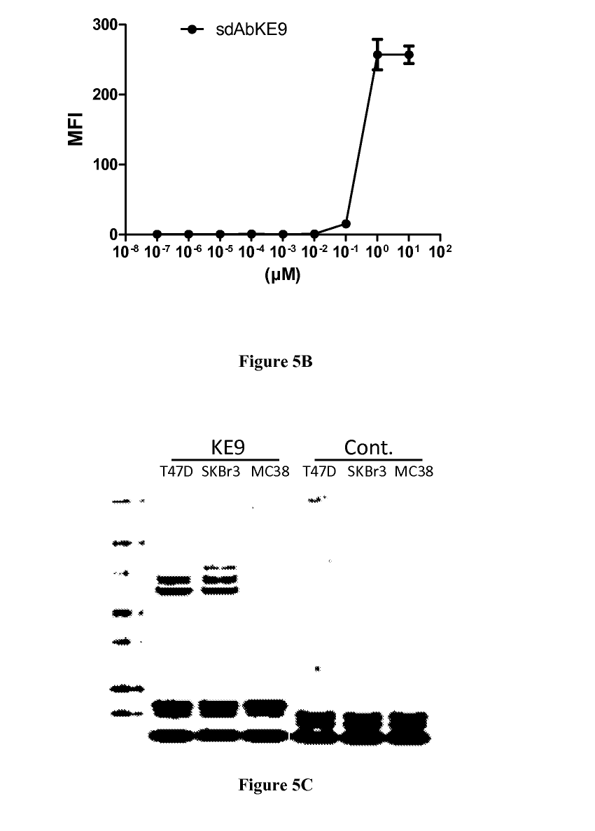

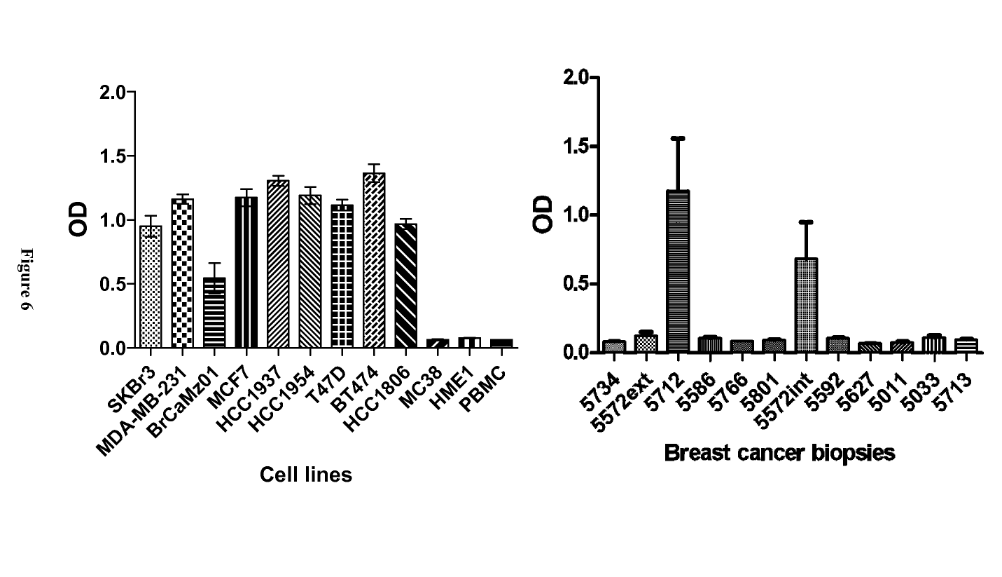

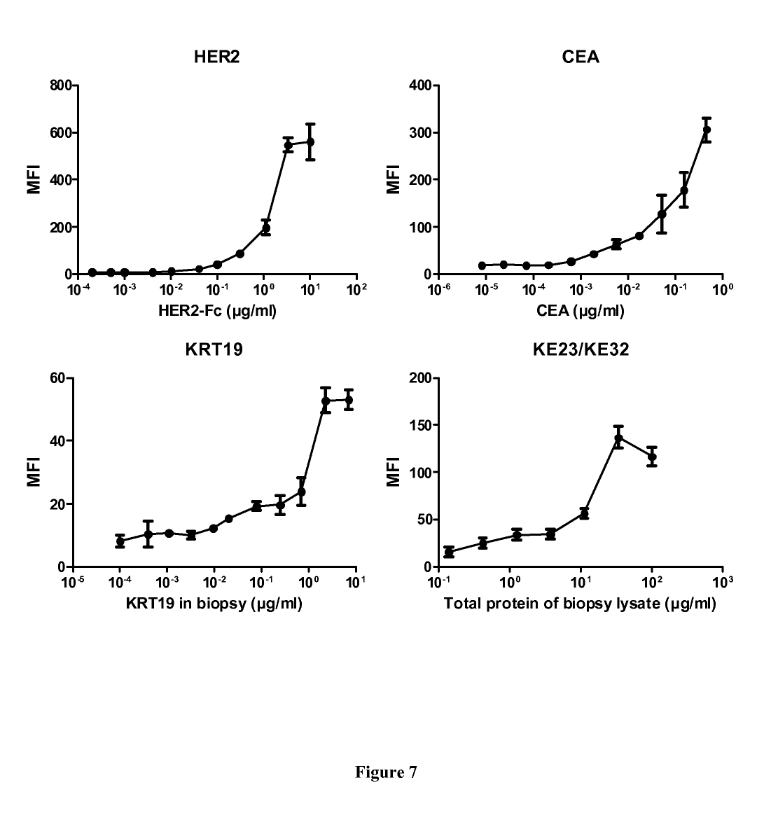

The experiments were conducted using the streptavidin/avidin based immobilization strategy and 50 nL of crude bacterial lysate per well. Detection of the bound antigen was performed using three different methods, namely using an anti-Nef mAb followed by a HPR-labeled secondary antibody or an Alexa-labeled secondary antibody, compared to a direct fluorescent labeling of the antigen (Nef-Alexa). As shown in FIG. 3A, the indirect labeling strategies yielded the best results, with a detection limit of 0.5 nM for enzymatic indirect labeling, 5 nM for fluorescent indirect labeling and 50 nM for the fluorescent direct labeling. A detection limit of 0.5 nM was measured on nitrocellulose slides using sandwich fluorescent detection. Streptavidin preincubation of slides further led to a significant decrease of background noise (FIG. 3D).

Application to Clinically Relevant Concentration of Cancer Biomarker:

To demonstrate that these strategies can be applied to high throughput diagnostic approaches, 0.5 μL of crude bacterial lysates containing in vivo biotinylated anti-CEA sdAbs were used in a bead based assay to detect soluble CEA in serial dilutions of crude cancer patient sera of known CEA concentration. Detection was performed using an enzymatic sandwich assay. As shown in FIG. 4, soluble CEA could be detected in all CEA-containing sera. The detection limit was established at 10 μM i.e. 2 ng/mL of soluble CEA. This concentration is below the value of circulating CEA in sera of normal donors (˜5 ng/mL in undiluted serum) and well below concentration of cancer patients (178-769 ng/mL).

Discussion:

Most antibody arrays developed to date are low density arrays relying on the use of pure preparation of intact monoclonal antibodies [24]. The requisite for high concentration of pure proteins is hindering the development of high density antibody arrays (in the 200-2000 μg/ml range). Recombinant antibodies such as scFv fragment offer an interesting alternative since this format is compatible with the generation of scFv libraries and high throughput selection methods such as phage or ribosome display. Unfortunately, those fragments are constituted by the association of two domains (VH and VL) which decreases their stability. Consequently, very high concentration of pure fragments (around 400 μg/mL) are often used to build microarrays [10, 25], which severely complicate the building process of high density antibody arrays.

In this study, we show that highly functional and sensitive arrays could be generated using non-purified affinity tagged single domain antibodies (sdAbs) as probes. These fragments are very easy to produce in E. coli, are compatible with cytoplasmic expression and are extremely stable. sdAbs were produced in 96 well plate format and successfully coupled, enriched and purified in a one-step procedure directly onto the support. Indeed, we demonstrate that extremely low amount, i.e. 0.5 to 0.05 μl (probably depending on the sdAb affinity) of crude bacterial lysate produced in three hours is sufficient to perform one assay. Such efficiency was achieved using strong and oriented immobilization on slide arrays or beads, through the use of directed cytoplasmic biotinylation of sdAbs for immobilization on streptavidin coated supports. Oriented immobilization based on modified with Ni²⁺-ions [14] or streptavidin[15, 16, 26] are examples of surface that have been successfully applied to generate planar protein arrays through specific coupling chemistries. However, to our knowledge, only purified monoclonal antibodies coupled with standard procedure such as carbodiimide and succinimide reactions are currently used for bead arrays.

High sensitivity and specificity are two crucial parameters for diagnostic arrays. In this case, the most efficient approach is the sandwich assay, using a pair of probes to specifically capture and detect the antigen of interest. In this case, non-purified sdAbs fulfill this need and can be efficiently immobilized using the biotin/streptavidin setting, to be used as capturing reagent. This method allowed a subnanomolar limit of detection (LOD) of a pure model antigen Nef using fluorescent and enzymatic detection methods. In a clinical setting, i.e. the detection of circulating CEA in sera of cancer patients, a picomolar LOD of CEA in crude serum was obtained with an enzymatic sandwich detection system. In the case of Nef detection, slides or beads as assay support yielded similar results. Of note, direct labeling of antigen with fluorophore was found very inefficient, and chemical sample biotinylation followed by detection with labeled streptavidin led to much higher signals, as already demonstrated by other studies [27]. Bead assays are especially suited for sandwich assays and can be directly compared to ELISA method [28], while requiring much smaller volumes of sample material. Beads can be coded by using various concentrations of fluorescent dye, or by some type of barcoding technology such as size of the bead. Consequently, bead assays can easily be multiplexed. Thus bead arrays are method of choice for low density antibodies array for clinical diagnosis [29, 30]. In this work we show that magnetic beads can efficiently be functionalized using a biotin-based sdAb immobilization. This approach would therefore be the method of choice for the development of cost-efficient sandwich-based antibody bead arrays for diagnostic.

In this work, we demonstrate that high sensitivities in the nanomolar range could be achieved with this setting for our model antigen on beads but also on planar arrays such as nitrocellulose arrays, clearly more adapted to high density arrays, and demonstrating the feasibility of using crude bacterial lysate to immobilize tagged sdAbs on slide in a high throughput screening compatible fashion. This approach can further be used for differential screening (i.e. using normal vs disease samples) of sdAb libraries enriched on disease material, potentially leading to the discovery of new biomarkers. We are currently applying this approach to isolate breast cancer specific sdAbs from libraries built using animals immunized with breast cancer biopsies.

### Example 2

### Use of Single Domain Antibody Array for Screening Breast Cancer Antigens

Material & Methods:

Production and Purification of sdAbs:

In vivo production of biotinylated sdAbs was performed as described in EXAMPLE 1.

ELISA on Epoxy Beads:

Antigens HER2-Fc (R & D systems) or Fc were immobilized on magnetic epoxy beads (Dynabeads, invitrogen) during 48 h at 4° C. following recommendation of the manufacturer. For ELISA, 2 μl of beads/well is used. Beads were blocked with 5% milk-PBS (MPBS) for two hours at RT. Beads were washed and incubated for 1 h at RT with 50 μl of 2% MPBS containing primary antibodies: in vivo biotinylated sdAbs-aCEA or -aHER2 at 10 μg/ml or HRP-conjugated anti-Fc mAb at 1 μg/ml. After three washes with PBS, beads with sdAbs were incubated with HRP-conjugated streptavidin (Jackson) (1 μg/ml) in 2% MPBS for one hour at RT. After three washes in PBS, bound secondary antibodies were detected using ABTS. Coloration was followed at 405 nm.

ELISA Using a Couple of sdAbs:

Streptavidin plates (Thermo scientific) were blocked with 5% milk-PBS (MPBS) for two hours at RT. Fifty μl/well of in vivo biotinylated sdAb at 10 μg/ml in 2% MPBS were incubated overnight at 4° C. Wells were washed and incubated for 1 h at RT with 50 μl of 2% MPBS containing cell (BT474, SK-BR-3, HCC1954, MCF7, MDA-MB-231, T47D, HCC1806, BRCA-Mz-01, HCC1937, HME1, MC38, PBMC) or biopsy (5734, 5772ext, 5712, 5586, 5766, 5801, 5572int, 5592, 5627, 5011, 5033, 5713) lysates at 100 μg/ml of total proteins. After three washes with PBS tween 0.1% and three washes in PBS, plates were incubated 1 h at RT with 50 μl/well of phage-containing supernatants diluted at ½ in 4% MPBS. Following three washes with PBS tween 0.1% and three washes in PBS plates were incubated with HRP-conjugated aM13 mAb (Pharmacia) at 1/5000 during 1 h at RT. After three washes with PBS Tween 0.1% and three washes in PBS, bound secondary antibodies were detected using ABTS. Coloration was followed at 405 nm.

Results:

To select binders against unknown breast cancer markers, llamas were immunized with breast cancer biopsy lysates and phage libraries were built and panned using various approaches. Several selections were performed on different samples including five different breast cancer cell lines and five biopsy lysates. Maxisorp plates and Epoxy magnetic beads were used alternatively as selection support to reduce the selection of non specific binders. In parallel, a depletion strategy was performed using lysates of a healthy human mammary epithelium cell line immortalized by telomerase over expression (hTERT-HME1). In some selection, an excess of binders obtained by previous selections was added as purified polyclonal sdAbs during the phage selection to favor the isolation of new binders against low-abundance or less immunogenic epitopes.

After two rounds of panning, a primary screening was performed by picking 188 clones for each strategy and performing a phage-sdAb ELISA on maxisorp-adsorbed lysates corresponding to each selection. Positive clones were selected to perform a secondary screening step against 9 breast cancer cell line lysates (BT474, SKBr3, HCC1954, MCF7, MDA-MB-231, T47D, HCC1806, BRCA-Mz-01, HCC1937), one healthy breast cell line (HME1) as control of cancer specificity, one mouse cell line (MC38) as control of human specificity and patient PBMC lysate as control of epithelial cells specificity. A similar secondary screening was performed in parallel against 12 breast cancer biopsy lysates (5734, 5772ext, 5712, 5586, 5766, 5801, 5572int, 5592, 5627, 5011, 5033, 5713). Around 200 clones were chosen according to their profile on the different lysates and were sequenced. A final set of 20 unique clones were selected for the diversity of their phage ELISA profile on breast cancer cell lines and biopsies.

Using this set of binders leading to various binding intensity on various lysates, it is possible to establish an antigenic profiling of unknown cancer samples. However, it is often desirable to know the antigen targeted by some binders. As a proof of concept, an approach was designed to elucidate the nature of the antigen recognized by one of these sdAbs. The clone KE9 leads to variable signals on different breast cancer cell line and biopsy lysates and no signal on mouse cell line (MC38) and PBMC (FIG. 5A). This result suggests than its antigen is expressed differentially in breast cancer cell lines and biopsies. By flow cytometry assay, this sdAb was shown to bind its antigen with an affinity in the range of 20 nM (FIG. 5B).

This sdAb was produced in the cytoplasm of E. coli in fusion with the avitag to allow an efficient and directed in vivo enzymatic biotinylation by BirA. The purified biotinylated sdAb was incubated with magnetic streptavidin beads before being incubated with two cell line lysates leading to strong signal by phage ELISA (T47D and SKBr3) and on a negative lysate (MC38) as control. Another negative control was performed by using these three lysates with an irrelevant sdAb (FIG. 5C). A SDS-PAGE analysis of the immunoprecipitation revealed several strong specific bands in the 50 kDa range. Mass spectrometry analysis of these bands identified the precipitated proteins as cytokeratin 19 (KRT19). The nature of KE9 antigen was confirmed by staining the KE9 immunoprecipate with an anti-KRT9 monoclonal antibody. A sandwich ELISA performed using the same mAb demonstrated that KE9 and this mAb do not share their epitope.

The identification of the antigens recognized by the specific binders is of interest and permits the identification of monoclonal antibodies to be used in a sandwich approach for sensitive and quantitative determination of the antigen concentration in various disease samples.

This step is however not compatible with high throughput selection of specific binders allowed by phage display technologies. To overcome this hurdle, we tested the possibility to set up a sandwich ELISA using a couple of sdAbs targeting different epitopes of the same unknown cancer marker. To determine a relevant sdAb couple, series of sandwich ELISA were performed with all binders previously selected on various lysates. A couple of sdAbs leading to robust signals (KE23 for capture and KE32 for detection) was chosen as a proof of concept. This couple of sdAbs was used to determine the presence of their antigen in various breast cancer cell line and biopsy lysates by sandwich sdAb ELISA (FIG. 6). The results suggest than this antigen is expressed differentially in breast cancers. In addition, no signal was obtained using a lysates of healthy breast cell line (HME1), mouse cell line (MC38) or human PBMC lysates, demonstrating the specificity of these sdAbs for human epithelial cancers.

### Example 3

### Use of Single Domain Antibody Array for Multiplexed Analysis

Methods:

Four types of CBA Functional Bead system (BD Biosciences) were used for the assay. The Functional Bead Conjugation Buffer Set was used for conjugation of streptavidin to beads following the recommendation of the manufacturer. For multiplexed assay, 1.5×10⁵ beads of each type were used per assay. The whole procedure was performed in the dark. Beads were coated with sdAbs individually and all types of beads were mixed for the rest of the procedure. Beads were blocked with 3% BSA PBS for 2 h at RT. Then beads were incubated with in vivo biotinylated sdAb (sdAb-aHer2, -aCEA, -aCK19, -KE23) at 10 μg/ml in 1% BSA PBS for 1 h at RT. After two washes with PBS, all beads type were mixed and incubated for 1 h at RT with serial dilution of sample containing patient serum with CEA, recombinant HER2-FC (R & D systems), lysate of a breast cancer biopsy (“biopsy 5712”). After two washes with PBS, beads were incubated for 1 h at RT with phage-sdAb KE32 at 10¹¹ phage/ml and anti-HER2 (Santa-Cruz, sc-74241), anti-CK19 (Santa-Cruz, sc-53258) and anti-CEA 35A7 antibody (kind gift of A. Pelegrin, Montpellier) at 2 μg/ml. After two washes with PBS, beads were incubated for 1 h at RT with anti-M13 mAb (Pharmacia). After two washes with PBS, beads were incubated for 1 h at RT with PE-conjugated goat anti mouse mAb (Santa-Cruz) at 1/200. Fluorescence was measured using a MACSQuant (Miltenyi) and results were analysed with the MACSQuant software. Negative (secondary antibody only) controls were carried out.

Results:

The straightforward selection approach as described in EXAMPLE 2 by phage display opens the possibility to rapidly select a variety of binders against various cancer samples and use the selected binders as binding unit to establish highly sensitive and quantitative diagnostic approaches. Therefore, we aimed at using the previously characterized binders to elaborate a multiplexed diagnostic assay for complex but precious samples such as biopsy lysates or patient serum. As a proof of concept, we decided to use previously isolated sdAbs against tumor markers HER2 and CEA (Behar, Chames et al. 2009), together with the anti-KRT19 sdAb and the couple of sdAbs KE23/32 (obtained in EXAMPLE 2) to build up a cytometric bead array assay. Four types of commercially available fluorescent beads were coated with streptavidin to immobilize in vivo biotinylated sdAbs on their surface in an orientated fashion as described in EXAMPLE 1. For this proof of concept, a complex sample was elaborated by mixing a patient serum containing a previously determined concentration of CEA, a purified recombinant HER2-Fc fusion, and the breast cancer biopsy lysate 5712 containing KRT19 and the unknown target. To evaluate the sensitivity of this approach, the precise concentration of KRT 19 contained in this lysate was first established by traditional sandwich ELISA by comparison with a standard curve obtained with the purified antigen. All four antigens were simultaneously detected using the corresponding mAb for known targets or by phage-sdAb (KE32) for the unknown target. As seen in FIG. 7, all antigens could be simultaneously detected with high sensitivity using a small amount (50 μL) of complex sample.

### REFERENCES

Throughout this application, various references describe the state of the art to which this invention pertains. The disclosures of these references are hereby incorporated by reference into the present disclosure.
[1] Ekins, R., Chu, F., Biggart, E., Multispot, multianalyte, immunoassay Ann Biol Clin (Paris) 1990, 48, 655-666.
[2] Ekins, R., Chu, F. W., Microarrays: their origins and applications. Trends Biotechnol 1999, 17, 217-218.
[3] Marcus, K., Joppich, C., May, C., Pfeiffer, K., et al., High-resolution 2DE. Methods Mol Biol 2009, 519, 221-240.
[4] Joos, T., Bachmann, J., Protein microarrays: potentials and limitations. Front Biosci 2009, 14, 4376-4385.
[5] Renberg, B., Nordin, J., Merca, A., Uhlen, M., et al., Affibody molecules in protein capture microarrays: evaluation of multidomain ligands and different detection formats. J Proteome Res 2007, 6, 171-179.
[6] MacBeath, G., Schreiber, S. L., Printing proteins as microarrays for high-throughput function determination. Science 2000, 289, 1760-1763.
[7] Haab, B. B., Dunham, M. J., Brown, P. 0., Protein microarrays for highly parallel detection and quantitation of specific proteins and antibodies in complex solutions. Genome Biol 2001, 2, RESEARCH0004.
[8] Rousserie, G., Sukhanova, A., Even-Desrumeaux, K., Fleury, F., et al., Semiconductor quantum dots for multiplexed bio-detection on solid-state microarrays. Crit Rev Oncol Hematol, 74, 1-15.
[9] Miller, J. C., Zhou, H., Kwekel, J., Cavallo, R., et al., Antibody microarray profiling of human prostate cancer sera: antibody screening and identification of potential biomarkers. Proteomics 2003, 3, 56-63.
[10] Carlsson, A., Wingren, C., Ingvarsson, J., Ellmark, P., et al., Serum proteome profiling of metastatic breast cancer using recombinant antibody microarrays. Eur J Cancer 2008, 44, 472-480.
[11] Ingvarsson, J., Wingren, C., Carlsson, A., Ellmark, P., et al., Detection of pancreatic cancer using antibody microarray-based serum protein profiling. Proteomics 2008, 8, 2211-2219.
[12] Sauer, G., Schneiderhan-Marra, N., Kazmaier, C., Hutzel, K., et al., Prediction of nodal involvement in breast cancer based on multiparametric protein analyses from preoperative core needle biopsies of the primary lesion. Clin Cancer Res 2008, 14, 3345-3353.
[13] Lyon, D. E., McCain, N. L., Walter, J., Schubert, C., Cytokine comparisons between women with breast cancer and women with a negative breast biopsy. Nurs Res 2008, 57, 51-58.
[14] Zhu, H., Bilgin, M., Bangham, R., Hall, D., et al., Global analysis of protein activities using proteome chips. Science 2001, 293, 2101-2105.
[15] Peluso, P., Wilson, D. S., Do, D., Tran, H., et al., Optimizing antibody immobilization strategies for the construction of protein microarrays. Anal Biochem 2003, 312, 113-124.
[16] Pavlickova, P., Knappik, A., Kambhampati, D., Ortigao, F., Hug, H., Microarray of recombinant antibodies using a streptavidin sensor surface self-assembled onto a gold layer. Biotechniques 2003, 34, 124-130.
[17] Honegger, A., Engineering antibodies for stability and efficient folding. Handb Exp Pharmacol 2008, 47-68.
[18] Hamers-Casterman, C., Atarhouch, T., Muyldermans, S., Robinson, G., et al., Naturally occurring antibodies devoid of light chains. Nature 1993, 363, 446-448.
[19] Gueorguieva, D., Li, S., Walsh, N., Mukerji, A., et al., Identification of single-domain, Bax-specific intrabodies that confer resistance to mammalian cells against oxidative-stress-induced apoptosis. FASEB J 2006, 20, 2636-2638.
[20] Muyldermans, S., Single domain camel antibodies: current status. J Biotechnol 2001, 74, 277-302.
[21] Baty, D., Chartier, M., Chames, P., Benichou, S., et al., WO/2009/066241 2009.
[22] Behar, G., Chames, P., Teulon, I., Cornillon, A., et al., Llama single-domain antibodies directed against nonconventional epitopes of tumor-associated carcinoembryonic antigen absent from nonspecific cross-reacting antigen. FEBS J 2009, 276, 3881-3893.
[23] Clarke, P., Mann, J., Simpson, J. F., Rickard-Dickson, K., Primus, F. J., Mice transgenic for human carcinoembryonic antigen as a model for immunotherapy. Cancer Res 1998, 58, 1469-1477.
[24] Knezevic, V., Leethanakul, C., Bichsel, V. E., Worth, J. M., et al., Proteomic profiling of the cancer microenvironment by antibody arrays. Proteomics 2001, 1, 1271-1278.
[25] Orchekowski, R., Hamelinck, D., Li, L., Gliwa, E., et al., Antibody microarray profiling reveals individual and combined serum proteins associated with pancreatic cancer. Cancer Res 2005, 65, 11193-11202.
[26] Andresen, H., Bier, F. F., Peptide microarrays for serum antibody diagnostics. Methods Mol Biol 2009, 509, 123-134.
[27] Zajac, A., Song, D., Qian, W., Zhukov, T., Protein microarrays and quantum dot probes for early cancer detection. Colloids Surf B Biointerfaces 2007, 58, 309-314.
[28] Elshal, M. F., McCoy, J. P., Multiplex bead array assays: performance evaluation and comparison of sensitivity to ELISA. Methods 2006, 38, 317-323.
[29] Schwenk, J. M., Gry, M., Rimini, R., Uhlen, M., Nilsson, P., Antibody suspension bead arrays within serum proteomics. J Proteome Res 2008, 7, 3168-3179.
[30] Rimini, R., Schwenk, J. M., Sundberg, M., Sjoberg, R., et al., Validation of serum protein profiles by a dual antibody array approach. J Proteomics 2009, 73, 252-266.

1. A method for preparing a single domain antibody (sdAb) microarray comprising the steps of:
i) providing a host cell capable of expressing a biotinylation enzyme
ii) transforming said host cell with a nucleic acid encoding for a fusion protein comprising a sdAb fused at its carboxy terminal end to a biotinylation peptide
iii) culturing said host cell in presence of biotin in such a way that said fusion protein is expressed, resulting in biotinylation of said fusion protein
iv) lysing said host cell as cultured at step iii) and
v) spotting the lysate obtained at step iv) on a solid support coated with an agent selected from the group consisting of avidin, streptavidin and/or a derivative of avidin or streptavidin.

2. The method according to claim 1 wherein the host cell can naturally express the biotinylation enzyme or was previously transformed with a nucleic acid encoding for the biotinylation enzyme.

3. The method according to claim 1 wherein the host cell is E. coli and the biotinylation enzyme is BirA.

4. The method according to claim 1, wherein the biotinylation peptide is a BirA substrate sequence tag.

5. The method according to claim 1, wherein the single domain antibody and the biotinylation peptide are fused directly or via a spacer.

6. The method according to claim 1, wherein the single domain antibody is specific for a cancer antigen or specific for a virus, a bacteria or a parasite associated target.

7. The method according to claim 1, wherein the solid support is selected from the group consisting of beads, plates, cuvettes, filters, and titer plates.

8. A single domain antibody (sdAb) microarray obtained by
i) providing a host cell capable of expressing a biotinylation enzyme
ii) transforming said host cell with a nucleic acid encoding for a fusion protein comprising a sdAb fused at its carboxy terminal end to a biotinylation peptide
iii) culturing said host cell in presence of biotin in such a way that said fusion protein is expressed, resulting in biotinylation of said fusion protein
iv) lysing said host cell as cultured at step iii) and
v) spotting the Mate obtained at step iv) on a solid support coated with an agent selected from the group consisting of avidin, streptavidin and/or a derivative of avidin or streptavidin.

9. The sdAb microarray according to claim 8, wherein said sdAb microarray comprises a sole type of sdAb directed against the same antigen, or with different kinds of sdAbs directed against various antigens.

10. The sdAb microarray according to claim 8 wherein the solid support of the sdAb array is a cytometric bead for use in flow cytometry.

11. A method for detecting a plurality of antigens comprising the steps of
providing a single domain antibody (sdAb) array by
i) providing a host cell capable of expressing a biotinylation enzyme
ii) transforming said host cell with a nucleic acid encoding for a fusion protein comprising a sdAb fused at its carboxy terminal end to a biotinylation peptide
iii) culturing said host cell in presence of biotin in such a way that said fusion protein is expressed, resulting in biotinylation of said fusion protein
iv) lysing said host cell as cultured at step iii) and
v) spotting the lysate obtained at step iv) on a solid support coated with an agent selected from the group consisting of avidin, streptavidin and/or a derivative of avidin or streptavidin,
contacting the array with a sample containing antigens, and
detecting antigens that bind to the array.


SEQUENCE LISTING (7 sequences)

SEQ ID NO: 1 - protein, 4 aa, Artificial
  Synthetic biotinylation peptide
AMKM

SEQ ID NO: 2 - protein, 15 aa, Artificial
  Synthetic biotinylation peptide
GLNDIFEAQKIEWHE

SEQ ID NO: 3 - DNA, 101 bp, Artificial
  Synthetic primer
tcagcaagcttaggatccgtgatgatgatggtggtgttcgtgccattcgattttctgagcctcgaagatgtcgttcagacctgcggccgctgaggagacag

SEQ ID NO: 4 - DNA, 20 bp, Artificial
  Synthetic primer
taatacgactcactataggg

SEQ ID NO: 5 - protein, 10 aa, Artificial
  Synthetic myc tag
EQKLISEEDL

SEQ ID NO: 6 - DNA, 79 bp, Artificial
  Synthetic primer
accgtctcctcagcggccgcagaacagaaactgatctctgaagaggacctgaacggtgagcagaagctcatttccgagg

SEQ ID NO: 7 - DNA, 87 bp, Artificial
  Synthetic primer
cgccaaaacagaagcttttagttgaggtcctcttcgctgatcaatttttgttcgccattcaaatcttcctcggaaatgagcttctgc